(19) United States
(12) Patent Application Publication
(10) Pub. No.: US 2023/0414650 A1
(43) Pub. Date: Dec. 28, 2023

(54) INHALATION AND TOPICAL USE OF A FORMULATION CONTAINING CYCLODEXTRIN, QUERCETIN AND ZINC, TO MITIGATE INFECTION BY ENVELOPED VIRUSES, SUCH AS HIV AND SARS-COV-2

(71) Applicants: Bhanu Pratap Jena, Bloomfield Hills, MI (US); Octavian Bucur, Waltham, MA (US)

(72) Inventors: Bhanu Pratap Jena, Bloomfield Hills, MI (US); Octavian Bucur, Waltham, MA (US)

(73) Assignee: Viron Therapeutics LLC, Boston, MA (US)

(21) Appl. No.: 17/207,250

(22) Filed: Mar. 19, 2021

Related U.S. Application Data
(60) Provisional application No. 63/019,312, filed on May 2, 2020.
(60) Provisional application No. 63/029,458, filed on May 23, 2020.

(51) Int. Cl. A61K 31/724; A61K 9/08; A61K 31/353; A61K 33/30

(52) U.S. Cl. CPC A61K 31/724; A61K 9/08; A61K 31/353; A61K 33/30

(57) ABSTRACT

The invention provides the use of a formulation containing cyclodextrine, quercetin and zinc at appropriate concentrations to mitigate infections by enveloped viruses, such as COVID-19 virus (SARS-CoV-2), influenza and HIV. While the different forms of cyclodextrin prevent the entry of coated viruses into host cells by extracting and sequestering cholesterol molecules at the virus envelope and at the host cell plasma membrane, the natural plant-based ionophore quercetin in the formulation enables cellular entry of zinc, inhibiting viral replication by altering polymerase activity in the host cell.

SUBSTANCE: This formulation is adapted for both surface use and introduction to a subject by dispersal, utilizing the known methods of measured nasal sprays or inhalers, wet wipes and medicated masks. The formulation is introduced in the form of an aqueous phosphate buffered saline pH 7.5 solution, containing 0.01% benzylkonium chloride as preservative and different concentrations of the active ingredients: 1-5% cyclodextrin; the flavonoid quercetin, a naturally occurring zinc ionophore at a concentration of 8 μg/ml; and 1 mg/ml zinc chloride. Depending on requirement, pH 2.5 citrate buffered aqueous will also be used, where the low pH serves as a preservative and a solvent for cyclodextrin. The active ingredient cyclodextrin (CD): is currently being used as an excipient in pharmaceutical products including in nasal sprays. The U.S. Food and Drug Administration (FDA) has approved the use of CDs since 2001. CDs were first employed in the the food industry in the 1970s, and since they have been used as food additives for carrying food-related lipophiles such as vitamins, aromas and colorants. The first pharmaceutical patent related to CDs and pharmaceutical applicability, was made in 1953 to serve as a complexing agents. Pharmaceutical products containing CDs comprise nasal spray, oral solutions, solid dosage forms, ocular and dermal formulations, suppositories, and parenteral solutions. Currently, more than 40 pharmaceutical products containing CDs are available in the market worldwide, and the vast majority of them utilize βCD and its derivatives having higher water solubility such as HPβCD, MβCD, and SBEβCD. Most of the βCD are also approved by the European Medical Agency for all human administration pathways. The active ingredients zinc and quercetin: Zinc is an essential trace element supporting growth, development and immune health. Zinc is also known to protect against viruses by inhibiting RNA binding, RNA synthesis, viral polyprotein cleavage, viral replication, and viral protease enzyme inactivation. Zinc however, needs to enter the host cell to protect against the virus. Quercetin, a naturally occurring plant-based over the counter zinc ionophore, enables the cellular entry of zinc to protect host cells against the virus. Furthermore, quercetin has shown therapeutic effects against influenza virus. Additionally, in silico modelling of the interactions between the SARS-CoV-2 viral spike protein and the epithelial cell angiotensin converting enzyme-2 (ACE2) protein, has identified quercetin from a database of 8,000 small molecule candidates of known drugs, metabolites, and natural products, as one of the top 5 most potent compounds for binding to the interface site, and disrupt initiation of viral infection.

### APPLICATION CROSS-REFERENCE

The instant Application claims priority to U.S. Provisional Patent Application Ser. No: 63/019312 filed on 2 May 2020; and, U.S. Provisional Patent Application Ser. No: 63/029458 filed on 23 May 2020, the entireties of which are incorporated by reference.

### BACKGROUND

### Technical Field

The present application generally relates to medicine, and more particularly to an inhalation and topical use of a formulation containing cyclodextrin, quercetin, and zinc to mitigate infection by enveloped viruses such as HIV and SARS-cov-2.

### Discussion of Art

Viruses enter hosts via the epithelium. The cell plasma membrane of skin and lung epithelia is the first line of defense and when breached, serves as the portal for viral entry into hosts. Studies in the past two decades report the various cell membrane binding and entry mechanisms utilized by viruses to infect. Irrespective of the different mechanisms involved in viral entry into host cells, the initiating critical process is binding of the virus to the cell plasma membrane. Without binding of virus to the cell plasma membrane, there would be no viral entry in to the host.

A large number of studies have established that binding of viruses to the cell plasma membrane is subjected to the presence of docking sites or receptors and their regulation by membrane lipid composition and distribution such as the establishment of domains called rafts. Our recent study involving cellular membrane biogenesis, demonstrate that changes in composition of membrane cholesterol, impacts both the chemistry and distribution of plasma membrane proteins and lipids, impacting cell function. In this study we report that cells exposed to an increasing concentration of methyl beta cyclodextrin (M-βCD) to deplete cholesterol from the cell plasma membrane demonstrate loss in the uptake of phosphotidyl serine by the cell plasma membrane [FIG. 1, FIG. 2], while the uptake of phosphatidylethanolamine remain unchanged. Similarly, the loss of cholesterol from the cell plasma membrane resulted in the depletion of membrane fusion proteins, such as syntaxin and SNAP25 from the plasma membrane, suggesting altered membrane fusogenicity [FIG. 3]. Therefore, changes to the chemistry of the epithelial cell plasma membrane via depletion of sterols/cholesterol by cyclodextrins (CDs) could dictate both the binding of the virus at the cell plasma membrane, and influence both the efficacy and potency of its entry into the host cell.

In agreement, recent studies demonstrate that depletion of plasma membrane cholesterol in host cells using M-βCD, significantly reduces entry of the pseudorabies and vaccinia virus into cells. Similarly, studies demonstrate that HIV infectivity is critically dependent on cholesterol.

Cholesterol microdomains, called lipid ‘rafts’, have been suggested in the cellular entry or infection of HIV, its assembly, and its release from infected cells. Studies further report that plasma membrane cholesterol is also required for a wide range of both bacterial and yeast infections. Furthermore, high-cholesterol diet impairs pulmonary host defense against gram-negative bacteria and Mycobacterium tuberculosis. Taken together, these results support that CD-mediated depletion of plasma membrane cholesterol in epithelial cells i.e., skin, nasal passage and lung epithelia in humans, using topological application, aerosol spray and nebulization, will mitigate both viral entry and secondary bacterial and yeast infections. Ds are a family of cyclic oligosaccharides constituted of a macrocyclic ring of glucose subunits joined by α-1,4 glycosidic bonds [FIG. 4]. CDs are used for improving the water-solubility and bioavailability of a wide range of drugs. The U.S. Food and Drug Administration (FDA) has approved the use of cyclodextrins since 2001. Cyclodextrins were first employed in the the food industry in the 1970s, and since they have been used as food additives for carrying food-related lipophiles such as vitamins, aromas and colorants. βCD has also been used as a cholesterol-reducing agent in food of animal origin such as milk and egg. The first pharmaceutical patent related to CDs and pharmaceutical applicability as complexing agents is dated 1953. Currently, cyclodextrins are employed in pharmaceutical products primarily to increase water solubility of poorly soluble drug formulations and to enhance drug bioavailabilities. Pharmaceutical products containing CDs comprise nasal spray, oral solutions, solid dosage forms, ocular and dermal formulations, suppositories, and parenteral solutions. Currently, more than 40 pharmaceutical products containing CDs are available on the market worldwide, and the vast majority of them utilize βCD and its derivatives having higher water solubility, such as HPβCD, MβCD, and SBEβCD. Most of the βCD are also approved by the European Medical Agency for all human administration pathways. CDs are used for example in tablets, aqueous parenteral solutions, nasal sprays and eye drop solutions. Examples of the use of cyclodextrins in medicines on the European market are (β-CD in cetirizine tablets and cisapride suppositories, γ-CD in minoxidil solution, and examples of the use of β-cyclodextrin derivatives are SBE-β-CD in the intravenous antimycotic voriconazole, HP-β-CD in the antifungal itraconazole, intravenous and oral solutions, and RM-β-CD in a nasal spray for hormone replacement therapy by 17β-estradiol. In Germany and Japan there are infusion products on the market, containing alprostadil (prostaglandin E1, PGE1) with α-CD. Cyclodextrins are currently not included in the European Commission Guideline on excipients in the label and package leaflet of medicinal products for human use. CDs themselves however, have never been used as an anti-viral, anti-bactricidal or anti-fungal drug until now, as presented here in this application.

### BRIEF SUMMARY OF THE INVENTION

In an embodiment of the invention there is used the non-toxic U.S. Food and Drug Administration (FDA)-approved excipient cyclodextrin as a drug in phosphate buffered solutions will allow the extraction of cholesterol molecules from the host cell membranes and enveloped virus membranes, altering their respective lipid and protein composition and distribution.

In an embodiment there is used the non-toxic U.S. Food and Drug Administration (FDA)-approved excipient cyclodextrin as a drug in phosphate buffered solutions, will allow the extraction of cholesterol molecules from the host cell membranes and enveloped virus membranes thus altering their respective lipid and protein composition and distribution, thus preventing virus entry into host cells.

In an embodiment there is used quercetin, a naturally occurring plant-based over the counter zinc ionophore, will enable the cellular entry of zinc to protect host cells against the virus by inhibiting RNA binding, RNA synthesis, viral polyprotein cleavage, viral replication, and viral protease enzyme inactivation.

In an embodiment, there is a combining of cyclodextrin, quercetin and zinc in a water-based soluble formulation, will prevent both viral entry and replication.

In an embodiment there is usage of an aerosol spray and nebulization of the combined cyclodextrin, quercetin and zinc as a aqueous phosphate buffered saline pH 7.5 solution containing 0.01% benzylkonium chloride as preservative will be used to protect the airways, including the lungs, from infection with enveloped viruses.

In an embodiment there is topical application of the combined cyclodextrin, quercetin and zinc in aqueous phosphate buffered saline pH 7.5 solution containing 0.01% benzylkonium chloride as preservative, will be used to protect body surface (skin) from infections with enveloped viruses.

In an embodiment there is an application to both sides of cellulose masks of the combined cyclodextrin, quercetin and zinc aqueous phosphate buffered saline pH 7.5 solution containing 0.01% benzylkonium chloride as preservative will further protect the airways, including the lungs, from infections with enveloped viruses. In such medicated masks, any airborne droplets containing the virus will be neutralized on contact with the medicated mask.

### BRIEF DESCRIPTION OF THE DRAWINGS

FIG. 1 is an illustration of cholesterol depletion from Min6 cells reducing the incorporation of phosphatidylserine from entering the cell plasma membrane.

FIG. 2 is an illustration that cholesterol depletion from Min6 cell plasma membranes has no significant effect on the incorporation of phosphatidylethanolamine into the cell plasma membrane.

FIG. 3. is an illustration that cholesterol depletion from Min6 cells alters the association-dissociation of both lipids and proteins in the cell plasma membrane.

FIG. 4 is an example of cyclodextrin structure and nomenclature for common cyclodextrins.

### DETAILED DESCRIPTION OF THE INVENTION

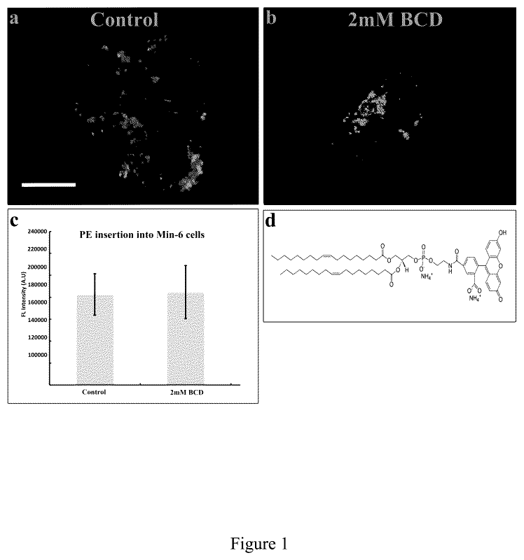

With reference to FIG. 1, cholesterol depletion from Min6 cells reduces the incorporation of phosphatidylserine from entering the cell plasma membrane. Estimation of fluorescent-PS in control Min6 cells (a), and in cells pre-exposed to 2 mM M βCD (b) and 5 mM M-βCD (c); scale bar-10 μm. Note the significant loss in fluorescent intensity upon M-βCD treatment of Min6 cells compared to controls (d). Chemical structure of PS lipid (e).

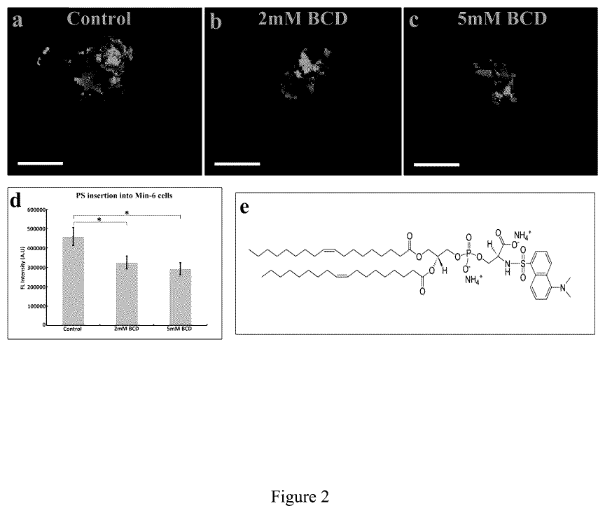

With reference to FIG. 2, cholesterol depletion from Min6 cell plasma membrane has no significant effect on the incorporation of phosphatidylethanolamine into the cell plasma membrane. Estimation of fluorescence in control (a), 2 mM M-βCD treated Min6 cells (b); scale bar=10 μm. Note no significant change in the presence of green fluorescent PE following M-βCD treatment (c). Chemical structure of PE lipid (d).

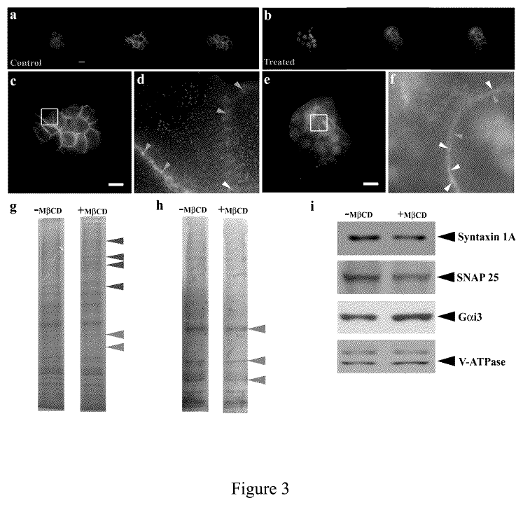

With reference to FIG. 3, cholesterol depletion from Min6 cells alters the association-dissociation of both lipids and proteins in the cell plasma membrane. Immunofluorescent staining of Min6 cells using antibody for two membrane-associated protein SNAP25 and Gai3, demonstrate their altered distribution following the extraction of membrane cholesterol using 5 mM M-bCD b,e,f compared to control cells a,c,d scale bar=10 μm. Control Min6 cells showing DAPI staining of the nucleus in blue (a), SNAP25 immunostaining in green, and Gai3 immunostaining in red. Note greater co-localization (white arrowheads show co-localization, while red arrowheads show separate green and red fluorescence) of SNAP25 and Gai3 at the cell plasma membrane in M-bCDtreated Min6 cells in the inset within white square in g presented in f, compared to controls d, scale bar=10 βm. Resolved membrane fractions from untreated (−M-bCD) and treated (+M-bCD) Min6 cells, demonstrate the loss (red arrowheads) or gain (green arrowheads) of proteins in coomassie g, and silver stained (h) SD S-PAGE. i, Western blot analysis of membrane proteins syntaxin, SNAP25, Gai3, and v-ATPase, further demonstrate changes in the presence of the proteins following extraction of cholesterol from the Min6 cell plasma membrane. Note while there is a loss of syntaxin and SNAP25, there is a gain in relative Gai3 and v-ATPase immunoreactivity in the plasma membrane of M-bCD-treated Min6 cells.

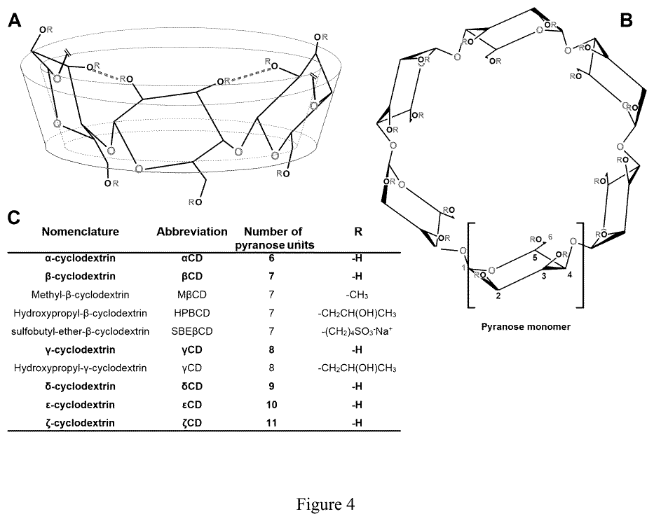

With reference to FIG. 4, side view (A); top view (B); nomenclature and chemical characteristics (C) found in most common cyclodextrins. The blue oxygens represent the etheric bonds 1-4 (inter-monomeric bonds), whereas, the red oxygens represents pyranose ether bonds 1-5 (intra-monomeric bonds). The green lines represent the intramolecular H-bonds forming in βCD when no substituent groups are present (R: —H).

Zinc is an essential trace element supporting growth, development and immune health. Zinc is also known to protect against viruses by inhibiting RNA binding, RNA synthesis, viral polyprotein cleavage, viral replication, and viral protease enzyme inactivation. Zinc however needs to enter the host cell to protect against the virus. Quercetin, a naturally occurring plant-based over the counter zinc ionophore, will enable the cellular entry of zinc to protect host cells against the virus. Furthermore, quercetin has shown therapeutic effects against influenza virus. Additionally, in silico modelling of the interactions between the SARS-CoV-2 viral spike protein and the epithelial cell angiotensin converting enzyme-2 (ACE2) protein, has identified quercetin from a database of 8,000 small molecule candidates of known drugs, metabolites, and natural products, as one of the top 5 most potent compounds for binding to the interface site, and disrupt initiation of viral infection.

Therefore, our CD formulation will utilize FDA approved concentrations of CDs, quercetin and zinc in buffered solutions to retain both high solubility and sterility. Mode of administration will be through aerosol spray and nebulization, and topical application on body surface using a water-based solution adsorbed to paper, cellulose or fabric. The topical application on body surfaces will including the face and neck, to mitigate envelop virus (such as COVID-19 virus (SARS-CoV-2 and HIV), bacteria and fungus infections.

The invention provides the use of a formulation containing cyclodextrine, quercetin and zinc, at appropriate concentrations to mitigate infections by enveloped viruses, such as COVID-19 virus (SARS-CoV-2), influenza and HIV. This formulation is adapted for both surface use and introduction to a subject by dispersal, utilizing the known methods of measured nasal sprays or inhalers, wet wipes and medicated masks. The formulation is introduced in the form of an aqueous phosphate buffered saline pH 7.5 solution, containing 0.01% benzylkonium chloride as preservative and different concentrations of the active ingredients: 1-5% cyclodextrin; the flavonoid quercetin, a naturally occurring zinc ionophore at a concentration of 8 μg/ml; and 1 mg/ml zinc chloride. Depending on requirement, pH 2.5 citrate buffered aqueous may or may not also be used, where the low pH serves as a preservative and a solvent for cyclodextrin.

While the different forms of cyclodextrin prevent the entry of enveloped viruses into host cells by extracting and sequestering cholesterol molecules at the virus coat and at the host cell plasma membrane, the natural plant-based ionophore quercetin in the formulation, enables cellular entry of zinc, which in turn inhibits viral replication by altering polymerase activity in the host cell.

Using the non-toxic U.S. Food and Drug Administration (FDA)-approved excipient cyclodextrin as a drug in phosphate buffered solutions, will allow the extraction of cholesterol molecules from enveloped virus membranes and the host cell membrane, altering their respective lipid and protein composition and distribution, preventing virus entry into host cells. Using quercetin, a naturally occurring plant-based over the counter zinc ionophore, will enable the cellular entry of zinc to protect host cells against the virus by inhibiting RNA binding, RNA synthesis, viral polyprotein cleavage, viral replication, and viral protease enzyme inactivation, among others. Combining cyclodextrin, quercetin and zinc in a water based soluble formulation, both viral entry and replication will be preventable. Aerosol spray and nebulization of the combined cyclodextrin, quercetin and zinc as a aqueous phosphate buffered saline pH 7.5 solution containing 0.01% benzylkonium chloride as preservative, will be used to protect the airways, including the lungs, from infections with enveloped viruses. Similarly, topical application of the combined cyclodextrin, quercetin and zinc in aqueous phosphate buffered saline pH 7.5 solution containing 0.01% benzylkonium chloride as preservative, will be used to protect body surface (skin) from infections with enveloped viruses. Additionally, application to both sides of cellulose masks of the combined cyclodextrin, quercetin and zinc aqueous phosphate buffered saline pH 7.5 solution containing 0.01% benzylkonium chloride as preservative, will further protect the airways, including the lungs, from infection with enveloped viruses. In such medicated masks, any airborn droplets containing the virus will be neutralized on contact with the medicated mask. This is the first direct use of cyclodextrin, quercetin and zinc as an anti-viral, anti-viral and anti-fungal drug.

This formulation is adapted for both surface use and introduction to a subject by dispersal, utilizing the known methods of measured nasal sprays or inhalers, wet wipes and medicated masks. The formulation is introduced in the form of an aqueous phosphate buffered saline pH 7.5 solution, containing 0.01% benzylkonium chloride as preservative and different concentrations of the active ingredients: 1-5% cyclodextrin; the flavonoid quercetin, a naturally occurring zinc ionophore at a concentration of 8 μg/ml; and 1 mg/ml zinc chloride. Depending on requirement, pH 2.5 citrate buffered aqueous will also be used, where the low pH serves as a preservative and a solvent for cyclodextrin. The active ingredient cyclodextrin (CD): is currently being used as an excipient in pharmaceutical products including in nasal sprays. The U.S. Food and Drug Administration (FDA) has approved the use of CDs since 2001. CDs were first employed in the food industry in the 1970s, and since they have been used as food additives for carrying food-related lipophiles such as vitamins, aromas and colorants. The first pharmaceutical patent related to CDs and pharmaceutical applicability, was made in 1953 to serve as a complexing agents. Pharmaceutical products containing CDs comprise nasal spray, oral solutions, solid dosage forms, ocular and dermal formulations, suppositories, and parenteral solutions. Currently, more than 40 pharmaceutical products containing CDs are available in the market worldwide, and the vast majority of them utilize βCD and its derivatives having higher water solubility such as HPβCD, MβCD, and SBEβCD. Most of the βCD are also approved by the European Medical Agency for all human administration pathways. The active ingredients zinc and quercetin: Zinc is an essential trace element supporting growth, development and immune health. Zinc is also known to protect against viruses by inhibiting RNA binding, RNA synthesis, viral polyprotein cleavage, viral replication, and viral protease enzyme inactivation. Zinc however, needs to enter the host cell to protect against the virus. Quercetin, a naturally occurring plant-based over the counter zinc ionophore, enables the cellular entry of zinc to protect host cells against the virus. Furthermore, quercetin has shown therapeutic effects against influenza virus. Additionally, in silico modelling of the interactions between the SARS-CoV-2 viral spike protein and the epithelial cell angiotensin converting enzyme-2 (ACE2) protein, has identified quercetin from a database of 8,000 small molecule candidates of known drugs, metabolites, and natural products, as one of the top 5 most potent compounds for binding to the interface site, and disrupt initiation of viral infection.

1-7. (canceled)

8. A method of reducing enveloped virus number comprising:
administering a solution of cyclodextrin.

9. The method of claim 8 wherein the solution is administered via nebulization.

10. The method of claim 8 wherein the solution is administered via inhaler.

11. The method of claim 8 wherein the solution is administered via atomized spray.

12. The method of claim 8 wherein the solution is buffered to a pH of 7.5 and contains 0.01% benzylkonium chloride.

13. The method of claim 8 wherein the solution is administered as part of a cellulose mask.

14. The method of claim 8 wherein the solution is phosphate buffered.

15. The method of claim 8 wherein the solution further comprises quercetin and zinc.
